(19) United States
(12) Patent Application Publication
(10) Pub. No.: US 2026/0273009 A1
(43) Pub. Date: Sep. 17, 2026

(54) Method of Enhancing Dihydromyricetin (DHM) Bioavailability

(71) Applicants: Jing LIANG, Rancho Palos Verdes, CA (US); Amy Sea SHAO, Bethesda, MD (US)

(72) Inventors: Jing LIANG, Rancho Palos Verdes, CA (US); Amy Sea SHAO, Bethesda, MD (US)

(21) Appl. No.: 19/679,334

(22) Filed: May 15, 2026

Related U.S. Application Data
(63) Continuation-in-part of application No. 18/669,211, filed on May 20, 2024, now Pat. No. 12,642,833.
Application No. 18/669,211 is a division of application No. 18/464,967, filed on Sep. 11, 2023, now Pat. No. 12,036,260.

(51) Int. Cl. A61K 36/72; A61K 36/258; A61K 36/28; A61K 36/752; A61K 36/882; A61P 25/20

(52) U.S. Cl. CPC A61K 36/72; A61K 36/258; A61K 36/28; A61K 36/752; A61K 36/882; A61P 25/20

(57) ABSTRACT

A composition and method for naturally enhancing the bioavailability, efficacy, and potency of dihydromyricetin (DHM) without altering its chemical structure. By combining DHM with lemon-derived components within defined pH ranges, superior solubility, stability, and oral exposure compared to DHM alone or citric acid formulations can be achieved. Preclinical data demonstrate increased plasma concentration, improved pharmacokinetics, and enhanced functional efficacy. This provides a reproducible and broadly applicable method for improving therapeutic outcomes in anxiety, sleep disturbance, cognitive impairment, and Alzheimer's disease.

### CROSS REFERENCES OF RELATED APPLICATIONS

This is a Continuation-In-Part of non-provisional application Ser. No. 18/669,211, filing date 05/20/2024, which is a divisional application of a non-provisional application Ser. No. 18/464,967, filing date of Sep. 11, 2023. These applications are incorporated by reference herein in their entireties.

### BACKGROUND OF THE PRESENT INVENTION

### Field of Invention

The present invention relates to the field of Traditional Chinese Medicine, and more particularly to a method of enhancing Dihydromyricetin (DHM) bioavailability which may be used for improving therapeutic outcomes in anxiety, sleep disturbance, cognitive impairment, and Alzheimer's disease.

### Description of Related Arts

Trouble sleeping is a problem that afflicts many people. Statistically, 4 out of 10 people have insomnia. A good night's sleep has become a luxury. In order to sleep well, many people choose to take sleeping pills or supplements.

Prescription medications may include benzodiazepines, nonbenzodiazepines, antidepressants, and melatonin receptors, which may cause grogginess during the day and induce addiction. Most over-the-counter medicines have antihistamines, which may have common side effects of daytime sleepiness, dizziness, confusion, a decline in thinking skills, and trouble peeing. However, all these medications are not meant for long-term use. Sleeping pills can be very damaging to the body, and it is easy to develop tolerance and addiction.

Therefore, the development of a product, which has a high efficacy for sleep while is safe for human use with minimal side effects without addiction, is urgently needed.

Dihydromyricetin (DHM) is a natural flavonoid with therapeutic potential for anxiety, alcohol use disorder, sleep disturbances, cognitive impairment, and Alzheimer's disease. However, its clinical utility has been limited by poor solubility, low stability, and inadequate oral bioavailability. Even when administered at higher doses, the plasma exposure of DHM is insufficient to achieve consistent efficacy. As a result, there is a strong need for a simple, natural, and safe method to improve DHM absorption and potency.

### SUMMARY OF THE PRESENT INVENTION

An object of the present invention is to provide a natural-based remedy which is non-additive, safe to use, and has long-lasting effect.

Another object of the present invention is to provide a composition containing Dihydromyricetin (DHM) with enhanced bioavailability and/or potency.

Another object of the present invention is to provide a composition containing DHM with enhanced solubility, stability, and bioavailability without any chemical modification of the DHM molecule itself.

Another object of the present invention is to provide a method of increasing bioavailability of DHM with natural lemon constituents.

Another object of the present invention is to provide a natural-based remedy to solve the problems of sleeping disorder, anxiety and sleep disturbances which is non-addictive and has a high level of DHM bioavailability.

According to a preferred embodiment of the present invention, a composition containing Dihydromyricetin (DHM) with enhanced bioavailability comprises:

0.05-0.5% w/v DHM in 10-40% v/v lemon-derived component in water, with a pH 2.0-6.0.

Preferably, the composition comprises 0.08-0.2% w/v DHM in 15-30% v/v lemon-derived component in water.

Preferably, an excipient selected from the group consisting of: sweetener, flavoring, stabilizer, and buffer is added.

Preferably, the lemon-derived component in water is a blended mixture of a whole fruit of lemon, including the outer skin and everything enclosed by the outer skin.

Preferably, the lemon-derived component in water is in the form of lemon juice in water and/or lemon juice powder. In addition, lemon essential oil may also be used.

Preferably, the composition containing Dihydromyricetin (DHM) with enhanced bioavailability is prepared by the steps of:
(a) juice one whole lemon to obtain 50 mL lemon-derived component in water;
(b) charge a 250-mL beaker with the lemon-derived component in water and a stir bar;
(c) add 200 mg DHM to the lemon-derived component in water while stirring; and

(d) add purified water q.s. to 200 mL (25% v/v lemon-derived component in water) to obtain a final product.

The final product has a pH of approximately 2.6-3.2 at 25° C. and a concentration of 1.0 mg/mL DHM (~0.1% w/v; ~3.1 mM).

In step (a), the lemon-derived component in water is a blended mixture of a whole fruit of lemon, including the outer skin and everything enclosed by the outer skin.

Preferably, the lemon-derived component in water is prepared by crushing and/or blending a whole lemon, including skin (such as zest and albedo), pulp (endocarp), and columella (central part). Then, the crushed and/or blended mixture is strained to remove seeds. In addition, excess pulp may also be removed.

The lemon-derived component in water may be prepared into lemon juice diluted in water and lemon juice powder. The lemon juice powder may be reconstituted in water when used.

According to a preferred embodiment of the present invention, a method of increasing bioavailability of Dihydromyricetin (DHM) comprises the steps of:
adding natural lemon constituent to DIM to obtain a composition of DHM with enhanced bioavailability,
wherein a concentration of DHM is 0.05-0.5% w/v and a concentration of natural lemon constituent is 10-40% v/v, and the DHM composition with enhanced bioavailability has a pH of 2.0-6.0.

The composition of DHM with enhanced bioavailability has complete solubility (i.e. no visible precipitate of DHM) at ambient conditions and under a microscope. The composition of DHM with enhanced bioavailability is clear without refrigeration for at least about one month.

The composition of DHM with enhanced bioavailability can promote good sleep and relaxation and provide other health benefits, such as memory and cognition improvements, and reducing anxiety.

### BRIEF DESCRIPTION OF THE DRAWINGS

FIG. 1 illustrates the results of DHM bioavailability of DHM with or without lemon extract according to Preferred Embodiment 1 of the present invention.

FIG. 2 illustrates the Plasma DHM concentration-time profiles (mean±SEM) according to Preferred Embodiment 2 of the present invention.

FIG. 3 illustrates the DHM-Lemon accelerated recovery from ethanol-induced LORR (mean±SEM) according to Preferred Embodiment 2 of the present invention.

### DETAILED DESCRIPTION OF THE PREFERRED EMBODIMENT

Dihydromyricetin (DHM) is an anti-alcohol intoxication medication. Dihydromyricetin (DHM, 1 mg/kg, i.p. injection) can counteract acute alcohol (EtOH) intoxication, and also withdrawal signs in rats including tolerance, increased anxiety and seizure susceptibility. DHM greatly reduced EtOH consumption in an intermittent voluntary EtOH intake paradigm in rats. At the cellular levels, DHM (1 μM) antagonized both acute EtOH-induced potentiation of GABA_{A}Rs and EtOH exposure/withdrawal-induced GABA_{A}R plasticity, including alterations in responsiveness of extra- and post-synaptic GABA_{A}Rs to acute EtOH, and most importantly, increases in GABA_{A}R α4 subunit expression in hippocampus and cultured neurons. DHM has been used as a hangover remedy and is an over-the-counter herbal remedy, which can help the body metabolize alcohol faster and treat alcohol use disorders. In particular, DHM has been used for easing headaches and providing protection to the liver. DHM has been used in Asia for thousands of years as a hangover cure and anti-intoxication medicine and is considered safe for humans even in massive doses.

According to the present invention, Dihydromyricetin (DHM), a bioflavonoid extracted from Hovenia or Rattan tea (vine), can counteract anxiety and depression via GABA_{A} receptor (GABA_{A}R) activity. DHM can counteract EtOH intoxication and withdrawal via GABA_{A}R activity. The activity of DHM is unique through combination with other remedies, such as resveratrol extracted from grape seed, genistein extracted from soy bean, daidzein extracted from Kudzu, and turmeric extracted from the ginger family. The levels of GABA_{A}R potentiation are different based on the different combinations with DHM.

DHM is a positive modulator of GABA_{A}R and the role of GABA_{A}R is to initiate sleep. Therefore DHM can be used to affect the levels of GABA_{A}R to relieve sleep disorder, and is an important ingredient used in the sleep aid supplement.

Hovenia or Rattan tea (vine) contains high component of dihydromyricetin (DHM), a bioflavonoid which can counteract anxiety and depression via GAB_{A}A receptor (GABA_{A}R) activity.

Hovenia extract refers to the extract prepared by seed of Hovenia dulcis Thunberg. Rattan extract refers to the extract prepared by the leaf of Rattan.

Lemon, Lisbon Lemons, contains flavonoids, natural compounds with antioxidant and anti-inflammatory properties. Major types of flavonoids found in lemon include hesperidin, quercetin (similar to DHM), rutin, naringin, and eriocitrin. Flavonoids in lemon are associated with potential benefits such as antioxidant protection, heart health support, anti-inflammatory effects, and immune system support.

It is worth mentioning that, according to the preferred embodiment of the present invention, the lemon-derived component is a blended mixture of the whole lemon fruit (Lisbon Lemons), including the outer skin and all the fruit and seeds inside. The lemon extract can greatly increase the bioavailability of DHM of the composition of the present invention.

According to one aspect of the present invention, a composition containing Dihydromyricetin (DHM) with enhanced bioavailability comprises: 0.05-0.5% w/v DHM in 10-40% v/v lemon-derived component in water, with a pH 2.0-6.0. Preferably, the composition comprises 0.08-0.2% w/v DHM in 15-30% v/v lemon-derived component in water. Preferably, the pH is 2.6-3.2 (e.g., by adjusting the percentage concentration of lemon-derived component in water to ~25% v/v).

Preferably, an excipient selected from the group consisting of: sweetener, flavoring, stabilizer, buffer and citrate is added to the composition.

Preferably, the lemon-derived component in water is a blended mixture of a whole fruit of lemon, including the outer skin and everything enclosed by the outer skin.

The lemon-derived component in water may be prepared to lemon juice in water (diluted with water), and lemon juice powder.

In some embodiments, lemon essential oil extracted from lemon peel may be added.

According to another aspect of the present invention, the composition containing Dihydromyricetin (DHM) with enhanced bioavailability is prepared by the steps of.
(a) juice one regular lemon to obtain 50 mL lemon-derived component in water;
(b) charge a 250-mL beaker with the lemon-derived component in water and a stir bar;
(c) add 200 mg DHM to the lemon-derived component in water while stirring; and
(d) add purified water q.s. to 200 mL (25% v/v lemon-derived component in water) to obtain a final product.

The final product has a pH of approximately 2.6-3.2 at 25° C. and a concentration of 1.0 mg/mL DHM (~0.1% w/v; ~3.1 mM).

In step (a), the lemon-derived component in water is a blended mixture of a whole fruit of lemon, including the outer skin and everything enclosed by the outer skin.

Preferably, in step (a), the lemon-derived component in water is prepared by crushing and/or blending a whole lemon, including skin (such as zest and albedo), pulp (endocarp), and columella (central part). Then, the crushed and/or blended mixture is strained to remove seeds and excess pulp.

The lemon-derived component in water may be prepared into lemon juice diluted in water and lemon juice powder. The lemon juice powder may be reconstituted in water when used.

According to another embodiment of the present invention, a method of increasing bioavailability of Dihydromyricetin (DHM) comprises the steps of:
adding natural lemon constituent to DIM to obtain a composition of DHM with enhanced bioavailability,
wherein a percentage concentration of DHM is 0.05-0.5% w/v and a percentage concentration of natural lemon constituent is 10-40% v/v, and the DHM composition with enhanced bioavailability has a pH of 2.0-6.0.

The composition of DHM with enhanced bioavailability has complete solubility (i.e. no visible precipitate of DHM) at ambient conditions and under a microscope. The composition of DHM with enhanced bioavailability is clear without refrigeration for at least about one month.

The natural lemon constituent is a natural whole lemon being prepared into lemon juice. Alternatively, the natural lemon constituent may refer to lemon juice in water, and/or lemon juice powder, with or without lemon essential oil.

The composition of DHM with enhanced bioavailability can promote good sleep and relaxation and provide other health benefits, such as memory and cognition improvements, and reducing anxiety.

By using the DHM composition with enhanced bioavailability at a dose of ≤1 mg/kg, an Cmax≥1,300 ng/mL and/or an AUC0-8 h≥6,500 ng·h/mL can be achieved.

### Preferred Embodiment 1

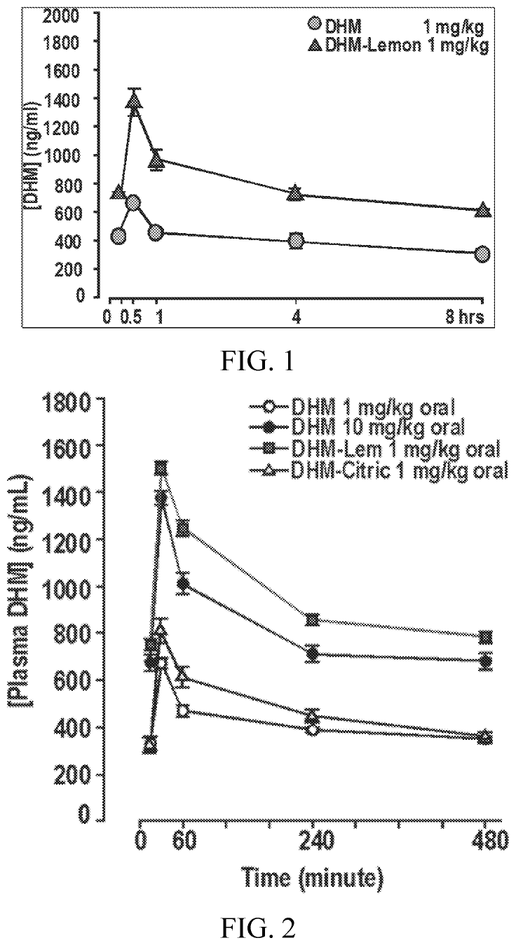

According to this embodiment, DHM absorption with or without lemon-derived component is studied by using DHM (300 mg DHM) and DHM-lemon (300 mg DHM and 150 mg lemon-derived component). The lemon-derived component is a blended mixture of a whole fruit of lemon, including an outer skin and everything enclosed by the outer skin The results show that the specific lemon-derived component of the present invention can dramatically increase DHM bioavailability and absorption, which is shown in FIG. 1 of the drawings.

In general, after DHM is orally administered, the DHM will go through the gastrointestinal tract and the bioavailability will decrease dramatically to about 4% or lower. So, the effect of orally administered DHM in improving the sleeping quality cannot be observed, or cannot be significantly or sufficiently observed. However, with the addition of lemon extract of the present invention, the bioavailability can be dramatically increased to about 40%. In other words, the combination effect of DHM and the lemon extract in the specific form and ratio of the present invention can dramatically increase the DHM bioavailability and its effect, such as showing a very strong, unexpected and effective result in improving the sleep quality.

It is worth mentioning that since the DHM (extractable from Hovenia or Rattan tea (vine)) and lemon are consumed by humans for more than hundreds of years, there's no report of side effect report based on literature search, and the DHM and lemon are ingredients safe to use without any significant side effect.

### Preferred Embodiment 2

A. Composition: The composition containing Dihydromyricetin (DHM) with enhanced bioavailability comprises: 0.05-0.5% w/v DHM in 10-40% v/v lemon juice in water, at pH 2.0-6.0. The preferred ranges include 0.08-0.2% w/v DHM with 15-30% v/v lemon juice. Optional excipients, including sweeteners, flavorings, stabilizers, and/or buffers (citrates), may be added.

B. Preparation method: Juice one regular lemon to obtain 50 mL with pulp/seeds removed. Charge a 250-mL beaker with the juice and a stir bar; add 200 mg DHM while stirring. Add purified water q.s. to 200 mL (25% v/v lemon juice). The resulting pH at 25° C. is about 2.6-3.2. The final concentration is 1.0 mg/mL DHM (~0.1% w/v; ~3.1 mM).

Specifically, the regular lemon refers to a whole lemon, including the outer skin and everything enclosed by the outer skin. The whole lemon is juiced by crushing or blending the whole lemon, with the seeds and excess pulp being removed.

C. Solubility, dissolution and Stability: The composition within the ranges of 0.05-0.5% w/v DHM in 10-40% v/v lemon juice and a pH of 2.0-6.0 exhibits complete solubility (that is, no visible precipitate) at ambient conditions and under a microscope. The solution can remain clear without refrigeration for at least 1 month

D. Storage condition: The solution can remain clear without refrigeration for at least 1 month. Storage above 80° C. is undesirable.

E. Pharmacokinetics Studies:

Comparative pharmacokinetics studies in mice are carried out.

Male mice are divided into four groups (total 32 mice, 8 per group). The four groups are given with a single oral dose of DHM 1 mg/kg, DHM 10 mg/kg, DHM-Lemon 1 mg/kg, or DHM-Citric 1 mg/kg. The Plasma DHM is measured at 0.5, 1, 4, and 8 h (mean±SEM, n=8/group). Key metrics are summarized in Table 1 below:

TABLE 1
Key Metrics of Pharmacokinetics Studies

| | AUC0-8 h | Cmax | |
|---|---|---|---|
| Group | (ng · h/mL) | (ng/mL) | Tmax (h) |
| DHM 1 mg/kg | 3166 | 673 | 0.5 |
| DHM 10 mg/kg | 6223 | 1376 | 0.5 |
| DHM-Lemon 1 mg/kg | 7402 | 1504 | 0.5 |
| DHM-Citric 1 mg/kg | 3731 | 814 | 0.5 |

Results: the composition with DHM-Lemon can increase both AUC and Cmax dramatically when compared to DHM alone (equal or 10× higher dose) and DHM-Citric, as shown in Table 1 and FIG. 2 of the drawings. Oral administration of DHM-Lemon at 1 mg/kg can significantly improve plasma exposure.

Compared to DHM 1 mg/kg, DHM-Lemon 1 mg/kg increases AUC by approximately 134% and increases Cmax by approximately 123%.

Compared to DHM 10 mg/kg, DHM-Lemon 1 mg/kg increases AUC by approximately 19% and Cmax by approximately 9%. The dosage of DHM-Lemon 1 mg/kg has a 10× lower dose compared to DHM 10 mg/kg.

Compared to DHM-Citric 1 mg/kg, DHM-Lemon 1 mg/kg increases AUC by approximately 98% and Cmax by approximately 85%, indicating a lemon-specific effect beyond citric acid alone. This indicates that the increase AUC and Cmax is a lemon-specific effect.

### F. Functional Efficacy Studies

Functional Efficacy in Ethanol-Induced the Loss of Righting Reflex (LORR) is studied. Ethanol (3 g/kg, i.p.) has the effect of prolonged time to wake from LORR. Co-administration with DHM-Lemon can reduce wake time (mean±SEM, n=8-9/group).

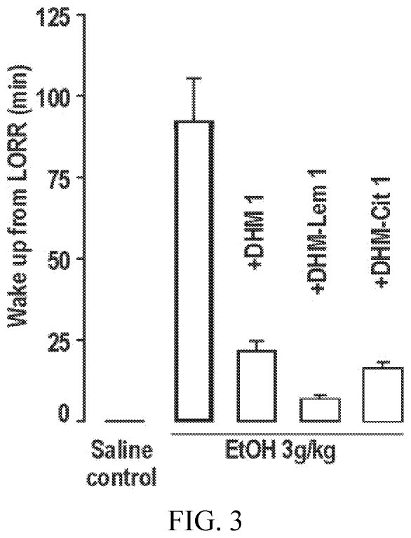

Referring to FIG. 3 of the drawings, the results of the five groups are illustrated. (Saline control=0; EtOH only=92.3±13.0 min; +DHM 1 mg/kg=21.6±3.2 min; +DHM-Lemon 1 mg/kg=6.9±1.1 min; +DHM-Citric 1 mg/kg=16.3 2.2 min). DHM-Lemon accelerates recovery by approximately 92.5% vs EtOH alone, outperforming DHM (approximately 76.6%) and DHM-Citric (approximately 82.4%).

The DHM-Lemon composition may be applied to the treatment of anxiety, sleep disturbances. The DHM-Lemon may also be applied to cognitive impairment and Alzheimer's disease. Because the DHM-Lemon formulation enhances DHM naturally without changing its chemical structure, it is possible to safely positioned as a nutraceutical, dietary supplement, or pharmaceutical preparation. The enhanced bioavailability allows for lower doses, reduced variability, and stronger efficacy, making the composition clinically and commercially valuable.

The present invention demonstrates that DHM's bioavailability and potency can be significantly enhanced using a simple, natural formulation with lemon components. Unlike chemical modification or synthetic derivatives, the method of the present invention retains the native DHM structure while solving the critical problem of poor absorption. The DHM-Lemon composition therefore represents a safe, effective, and innovative approach to unlock DHM's full therapeutic potential.

One skilled in the art will understand that the embodiment of the present invention as shown in the drawings and described above is exemplary only and not intended to be limiting.

It will thus be seen that the objects of the present invention have been fully and effectively accomplished. It embodiments have been shown and described for the purposes of illustrating the functional and structural principles of the present invention and is subject to change without departure from such principles. Therefore, this invention includes all modifications encompassed within the spirit and scope of the following claims.

What is claimed is:

1. A method of enhancing dihydromyricetin bioavailability, comprising the steps of:
adding lemon-derived component in water to dihydromyricetin (DHM) to obtain a DHM composition with enhanced bioavailability;
wherein a pH of the DHM composition with enhanced bioavailability is based on a percentage concentration of the lemon-derived component and the DHM,
wherein the DHM has a concentration of 0.05-0.5% w/v, the lemon-derived component has a concentration of 10-40% v/v, and the pH of the DHM composition with enhanced bioavailability is 2.0-6.0.

2. The method according to claim 1, wherein the DHM has a concentration of 0.08-0.2% w/v, the lemon-derived component has a concentration of 15-30% v/v, and the pH of the DHM composition with enhanced bioavailability is 2.6-3.2.

3. The method according to claim 2, wherein lemon-derived component is selected from the group consisting of lemon juice in water, lemon juice powder, a mixture of lemon juice in water and lemon juice powder, a mixture of lemon juice in water and lemon essential oil, and a mixture of lemon juice powder and lemon essential oil.

4. The method according to claim 1, wherein the lemon-derived component is a blended or crushed mixture of a whole fruit of lemon, including an outer skin and everything enclosed by the outer skin, with seeds being removed.

5. The method according to claim 2, wherein the lemon-derived component is a blended or crushed mixture of a whole fruit of lemon, including an outer skin and everything enclosed by the outer skin, with seeds being removed.

6. The method according to claim 4, further comprising the steps of: adding a pharmaceutically acceptable excipient selected from the group consisting of: sweetener, flavoring, pectin, stabilizer, buffer and citrate.

7. The method according to claim 5, further comprising the steps of: adding a pharmaceutically acceptable excipient selected from the group consisting of: sweetener, flavoring, pectin, stabilizer, buffer and citrate.

8. The method according to claim 2, wherein a dose of the DHM composition with enhanced bioavailability is ≤1 mg/kg is capable of a resulting Cmax≥1,300 ng/mL, a resulting AUC0-8 h≥6,500 ng·h/mL, or a resulting Cmax≥1,300 ng/mL and a resulting AUC0-8 h≥6,500 ng·h/mL.

9. The method according to claim 4, wherein a dose of the DHM composition with enhanced bioavailability is ≤1 mg/kg is capable of a resulting Cmax≥1,300 ng/mL, a resulting AUC0-8 h≥6,500 ng·h/mL, or a resulting Cmax≥1,300 ng/mL and a resulting AUCO-8 h≥6,500 ng·h/mL.

10. The method according to claim 5, wherein a dose of the DHM composition with enhanced bioavailability is ≤1 mg/kg is capable of a resulting Cmax≥1,300 ng/mL, a resulting AUCO-8 h≥6,500 ng·h/mL, or a resulting Cmax≥1,300 ng/mL and a resulting AUCO-8 h≥6,500 ng·h/mL.

11. The method according to claim 2, wherein an effective amount of the DHM composition with enhanced bioavailability is capable of treating anxiety and sleep disturbance.

12. The method according to claim 9, wherein an effective amount of the DHM composition with enhanced bioavailability is capable of treating anxiety and sleep disturbance.

13. The method according to claim 10, wherein an effective amount of the DHM composition with enhanced bioavailability is capable of treating anxiety, sleep disturbance, cognitive impairment, or Alzheimer's disease.

14. A DHM composition with enhanced bioavailability, comprising: 0.05-0.5% w/v DHM in 10-40% v/v lemon-derived component in water, wherein a pH of the DHM composition with enhanced bioavailability is 2.0-6.0 based on the percentage concentration of DHM and lemon-derived component in water.

15. The DHM composition with enhanced bioavailability according to claim 14, wherein the percentage concentration of DHM is 0.08-0.2% w/v and the percentage concentration of lemon-derived component in water is 15-30% v/v, and the pH of the DHM composition with enhanced bioavailability is 2.6-3.2.

16. The DHM composition with enhanced bioavailability according to claim 15, wherein the lemon-derived component in water is a blended or crushed mixture of a whole fruit of lemon, including an outer skin and everything enclosed by the outer skin, with seeds being removed and diluted with water.

17. The DHM composition with enhanced bioavailability according to claim 16, wherein the lemon-derived component in water is prepared into the form of lemon juice in water or lemon juice powder.

18. The DHM composition with enhanced bioavailability according to claim 16, further comprising a pharmaceutically acceptable excipient selected from the group consisting of: sweetener, flavoring, pectin, stabilizer, buffer and citrate.

19. The DHM composition with enhanced bioavailability according to claim 14, wherein a dose of the DHM composition with enhanced bioavailability is ≤1 mg/kg is capable of a resulting Cmax≥1,300 ng/mL, a resulting AUC0-8 h≥6,500 ng·h/mL, or a resulting Cmax≥1,300 ng/mL and a resulting AUC0-8 h≥6,500 ng·h/mL.

20. The DHM composition with enhanced bioavailability according to claim 16, wherein a dose of the DHM composition with enhanced bioavailability is ≤1 mg/kg is capable of a resulting Cmax≥1,300 ng/mL, a resulting AUCO-8 h≥6,500 ng·h/mL, or a resulting Cmax≥1,300 ng/mL and a resulting AUC0-8 h≥6,500 ng·h/mL.
